(19) United States
(12) Patent Application Publication
(10) Pub. No.: US 2026/0175035 A1
(43) Pub. Date: Jun. 25, 2026

(54) IMPLANTABLE MEDICAL DEVICE TO DETECT HEALTH EVENT

(71) Applicant: Medtronic, Inc., Minneapolis, MN (US)

(72) Inventors: Joshua J. Blauer, White Bear Township, MN (US); Kevin L. Sack, New Brighton, MN (US); Kaileigh E. Hayden, Mahtomedi, MN (US)

(21) Appl. No.: 19/125,020

(22) PCT Filed: Oct. 11, 2023
(86) PCT No.: PCT/US2023/076611

§ 371 Date: Apr. 28, 2025

Related U.S. Application Data
(60) Provisional application No. 63/381,455, filed on Oct. 28, 2022.

(51) Int. Cl. A61N 1/37; A61B 5/0245; A61N 1/362; A61N 1/368

(52) U.S. Cl. CPC A61N 1/3702; A61B 5/0245; A61N 1/3622; A61N 1/3627; A61N 1/368

(57) ABSTRACT

An example system includes a plurality of electrodes; a motion sensor configured to detect motion; therapy generation circuitry electrically coupled to one or more of the plurality of electrodes; and processing circuitry configured to: control the therapy generation circuitry to deliver an electrical stimulation therapy pacing protocol to a heart via one or more of the plurality of electrodes over a period of time to increase heartrate during the period of time to at least a target heartrate; and determine an indication of heart failure based, on the detected, motion during the period of time.

This application is an international application with provisional priority of U.S. Provisional Patent Application. No. 63/381,455 , filed 28 Oct. 2022, the entire contents of which are incorporated herein by reference.

### TECHNICAL FIELD

The disclosure relates to medical devices, and more particularly to the detection of a health event, such as onset or progression of heart failure, by the medical devices.

### BACKGROUND

An implantable pacemaker may deliver pacing pulses to a patient's heart and monitor conditions of the patient's heart. In some examples, the implantable pacemaker comprises a pulse generator and one or more electrical leads. The pulse generator may, for example, be implanted in a small pocket in the patient's chest. The electrical leads may be coupled to the pulse generator, which may contain circuitry that generates pacing pulses and/or senses cardiac electrical activity. The electrical leads may extend from the pulse generator to a target site (e.g., an atrium and/or a ventricle) such that electrodes at the distal ends of the electrical leads are positioned at the target site. The pulse generator may provide electrical stimulation to the target site and/or monitor cardiac electrical activity at the target site via the electrodes.

Other implantable pacemakers are configured to be implanted entirely within a chamber of the heart. Such pacemakers may be referred to as intracardiac pacing devices or leadless pacing devices, and may include one or more electrodes on their outer housings to deliver therapeutic electrical signals and/or sense intrinsic depolarizations of the heart. Such pacemakers may be positioned within or outside of the heart and, in some examples, may be anchored to a wall of the heart via a fixation mechanism.

### SUMMARY

In general, this disclosure is directed to techniques for an implantable medical device to detect deterioration of cardiac function based on motion of the heart during a period of elevated heartrate. For example, the implantable medical device may create a virtual stress test, through an elevated pacing rate protocol, to expose dysfunction in the heart that may be sensed mechanically using the motion sensor of a pacemaker to provide effective detection of patients progressing into heart failure (HF). Implantable medical device may also perform a sweep of atrioventricular (AV) intervals during when the heartrate is at least a target threshold to provide effective detection of patients progressing into HF. Since testing, in accordance with the devices and techniques described herein, may occur between office visits, deterioration of cardiac function may be detected sooner and more efficiently.

In one example, a system comprising a plurality of electrodes; a motion sensor configured to detect motion; therapy generation circuitry electrically coupled to one or more of the plurality of electrodes; and processing circuitry configured to: control the therapy generation circuitry to deliver an electrical stimulation therapy pacing protocol to a heart via one or more of the plurality of electrodes over a period of time to increase heartrate during the period of time to at least a target heartrate; and determine an indication of heart failure based on the detected motion during the period of time.

In another example, a system comprising a plurality of electrodes; a motion sensor configured to detect motion; therapy generation circuitry electrically coupled to one or more of the plurality of electrodes; and processing circuitry configured to: determine, based on the detected motion, a heartrate of a heart is at least a target threshold; in response to determining the heartrate is at least the target threshold, control the therapy generation circuitry to deliver, via one or more of the plurality of electrodes, cardiac pacing to the heart at a plurality of atrioventricular intervals over a period of time; and determine an indication of heart failure based on the detected motion during the period of time.

In another example, a method comprising delivering, by circuitry and via one or more of a plurality of electrodes, an electrical stimulation therapy pacing protocol to a heart to increase heartrate during a period of time to at least a target heartrate; detecting, by a motion sensor, motion during the pacing protocol during the period of time; and determining, by the circuitry, an indication of heart failure of the heart based on the detected motion during the period of time.

In another example, a system comprising a plurality of electrodes; a motion sensor configured to detect motion; therapy generation circuitry electrically coupled to one or more of the plurality of electrodes; and processing circuitry configured to: control the therapy generation circuitry to deliver electrical pacing to a heart via one or more of the plurality of electrodes over a period of time at least one target heartrate; and determine an indication of heart failure based on a mean motion sensor amplitude detected during the period of time.

In another example, a method comprising delivering, by circuitry and via one or more of a plurality of electrodes, an electrical pacing to a heart over a period of time of at least a target heartrate; detecting, by a motion sensor, a mean motion sensor amplitude during the pacing protocol during the period of time; and determining, by the circuitry, an indication of heart failure based on the detected a mean motion sensor amplitude during the period of time.

This summary is intended to provide an overview of the subject matter described in this disclosure. It is not intended to provide an exclusive or exhaustive explanation of the methods and systems described in detail within the accompanying drawings and description below. The details of one or more aspects of the disclosure are set forth in the accompanying drawings and the description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims,

FIG. 1 is a conceptual diagram illustrating an example pacing device implanted within a patient.

FIG. 2 is a conceptual illustration of an example configuration of the pacing device of FIG. 1.

FIG. 3 is a perspective drawing illustrating another example configuration of a pacing device.

FIG. 4A is a conceptual block diagram of an example implantable medical device, which may be implemented in or as the pacing device of FIGS. 1-2, or the pacing device of FIG. 3.

FIG. 4B is an example of a motion sensor signal that may be acquired over a cardiac cycle by a motion sensor included in the pacing devices or implantable medical device of FIGS. 1-4A.

FIGS. 5A-5C illustrate graphs showing examples of relationships of hemodynamic information indicative of the effect of heartrate on discoverability of heart failure.

FIGS. 6A-6D are plots of motion sensor amplitude root mean square (RMS) vs. measured ejection fraction for a group of study animal hearts.

FIG. 7 is a flow diagram illustrating an example process executable by an implantable medical device including the pacing devices or implantable medical device of FIGS. 1-4.

### DETAILED DESCRIPTION

Brady pacing provides a life preserving therapy for bradycardic patients.

However, 10 to 15% of patients requiring frequent right ventricular pacing experience the onset, or worsening, of heart failure (HF), e.g., pacing induced cardiomyopathy (PICM). Early detection of changes in the tissue properties, such as the onset of HF or worsening HF. would permit intervention before significant damage is done.

In general, this disclosure describes example techniques related to creating a virtual stress test, through an elevated pacing rate protocol, to expose dysfunction in the heart that may be sensed mechanically using the motion sensor of a pacemaker to provide effective detection of patients progressing into HF. In some examples, in addition to or instead of elevated pacing rates, the techniques may include a sweep of AV intervals that may similarly expose dysfunction in the heart that may be sensed mechanically using the motion sensor of a pacemaker. Values of one or more features of the motion signal, determined according to the techniques described herein, may correspond to cardiac mechanical function and HF status.

It is believed that myocardial contractility in a normal healthy heart would increase with heartrate increase due to physiological autonomic regulation. Consequently, absence of contractility increases could indicate pathology. Additionally, some pathological manifestations of lusitropic effects, e.g., extended relaxation, would be undetectable at low rates. At higher rates, when the diastolic time window is shortened, these pathologies would become evident. Additionally, pacing the heart at an elevated rate allows for motion sensor data collection at a relatively constant and normalized heartrate (in contrast to natural sinus rhythm, which can vary), and better comparability between or among accelerometer data sets taken at different times,

A healthy individual has a degree of “cardiac reserve”, untapped physiological resources that the cardiovascular system can exploit to maintain cardiac function at normal rates, pressures and outputs. In contrast, a patient with HF has impaired/depleted cardiac reserves, and the cardiovascular system would not be able to compensate as easily. When rates are elevated and the heart is forced to adapt, the lack of cardiac reserves would become evident. This would be measurable through mechanical motion via one or more motion sensors on an implantable medical device, such as an intracardiac leadless pacemaker.

The device, system, and/or techniques described may help detect deterioration of cardiac function sooner and more efficiently as this testing may occur between clinic visits, which may provide an earlier warning of trouble to a clinician. Based on motion metric values, the device, system, and/or techniques may also provide feedback on how effectively clinical interventions for HF are working, such as whether are they providing benefit as reflected in the mechanical functioning of the heart. Such feedback, if provided to the patient, may also enhance patient compliance with such interventions, e.g., medication. In addition, an indication of HF may be determined during routine capture management or periods of naturally high heart rate, e.g., due to exercise, which may result in little to no additional risk to patients associated with higher pacing rates, while obtaining vital information that may extend the length and quality of life of the patient.

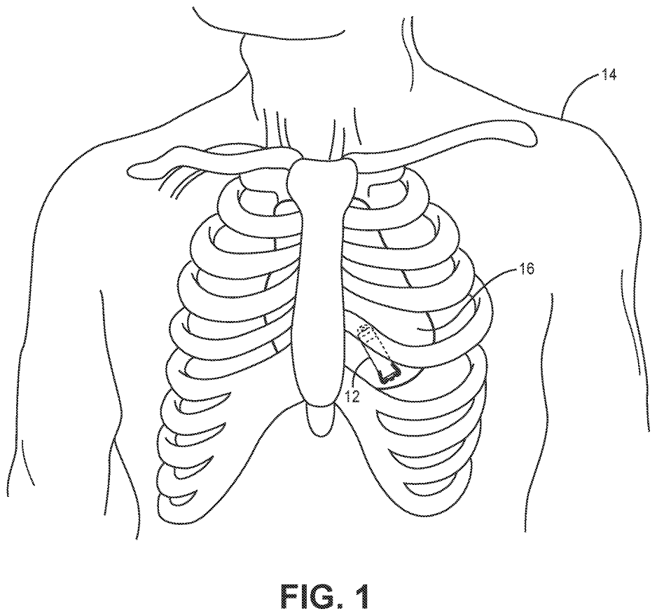

FIG. 1 is a conceptual diagram illustrating an example pacing device 12 implanted within a patient 14. Pacing device 12 is an example of an implantable medical device that may be fixed to heart 16 to provide electrical signals via electrodes to heart 16 and facilitate detection of motion of heart 16 as described herein. Pacing device 12 may be, for example, an implantable leadless pacing device that is configured for implantation entirely within one of the chambers of heart 16, and that provides electrical signals to heart 16 via electrodes carried on the housing of pacing device 12.

Pacing device 12 is generally described as being implanted within a chamber of heart 16 as an intracardiac pacing device. In other examples that are consistent with aspects of this disclosure, pacing device 12 may be affixed to an external surface of heart 16, such that pacing device 12 is disposed outside of heart 16 but can pace a desired chamber. In one example, pacing device 12 is affixed to an external surface of heart 16, and one or more components of pacing device 12 may be in contact with the epicardium of heart 16. Pacing device 12 may be affixed to a wall of a ventricle of heart 16, or other chamber, via one or more fixation elements (e.g., tines, helix, etc.) that penetrate the tissue. These fixation elements may secure pacing device 12 to the cardiac tissue and retain an electrode (e.g., a cathode or an anode) in contact with the cardiac tissue. Pacing device 12 may be implanted at or proximate to the apex of the heart. In other examples, a pacing device may be implanted at other ventricular locations, e.g., on the free-wall or septum, an atrial location, or any location on or within heart 16. Being fixed to heart 16 may facilitate detection of motion of the heart by pacing device 12.

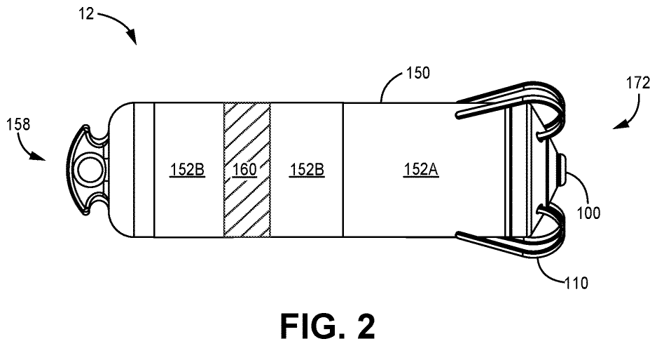

FIG. 2 is a conceptual illustration of an example configuration of pacing device 12. Pacing device 12 is configured to be implanted within a chamber of a heart of a patient, e.g., to monitor electrical activity of the heart and/or provide electrical therapy to the heart. In the example shown in FIG. 2, pacing device 12 includes outer housing 150, a plurality of fixation tines 110 and electrodes 100 and 160.

Outer housing 150 has a size and form factor that allows pacing device 12 to be entirely implanted within a chamber of a heart of a patient. In some examples, outer housing 150 may have a cylindrical (e.g., pill-shaped or capsule-shaped) form factor. Pacing device 12 may include a fixation mechanism configured to fix pacing device 12 to cardiac tissue. For example, in the example shown in FIG. 2, pacing device 12 includes fixation tines 110 extending from housing 150 and configured to engage with cardiac tissue to substantially fix a position of housing 150 within the chamber of the heart 16. Fixation tines 110 are configured to anchor housing 150 to the cardiac tissue such that pacing device 12 moves along with the cardiac tissue during cardiac contractions. Fixation tines 110 may be fabricated from any suitable material, such as a shape memory material (e.g., Nitinol). Although pacing device 12 includes a plurality of fixation tines 110 that are configured to anchor pacing device 12 to cardiac tissue in a chamber of a heart, in other examples, pacing device 12 may be fixed to cardiac tissue using other types of fixation mechanisms, such as, but not limited to, barbs, coils, and the like.

Housing 150, also referred to as an elongated housing, houses electronic components of pacing device 12, e.g., sensing circuitry for sensing cardiac electrical activity via electrodes 100 and 160 and therapy generation circuitry for delivering electrical stimulation therapy via electrodes 100 and 160. Electronic components may include any discrete and/or integrated electronic circuit components that implement analog and/or digital circuits capable of producing the functions attributed to pacing device 12 described herein. In some examples, housing 150 may also house components for sensing other physiological parameters, such as acceleration, pressure, sound, and/or impedance.

Additionally, housing 150 may also house a memory that includes instructions that, when executed by processing circuitry housed within housing 150, cause pacing device 12 to perform various functions attributed to pacing device 12 herein. In some examples, housing 150 may house communication circuitry that enables pacing device 12 to communicate with other electronic devices, such as a medical device programmer. In some examples, housing 150 may house an antenna for wireless communication. Housing 150 may also house a power source, such as a battery. Housing 150 can be hermetically or near-hermetically sealed in order to help prevent fluid ingress into housing 150.

Pacing device 12 is configured to sense electrical activity of the heart and deliver electrical stimulation to the heart via electrodes 100 and 160. Electrode 100 and/or electrode 160 may be mechanically connected to housing 150. As another example, electrode 100 and/or electrode 160 may be defined by an outer portion of housing 150 that is electrically conductive. For example, electrode 160 may be defined by a conductive portion of housing 150. In some examples, electrode 160 may serve as an anode and/or a return electrode, and electrode 100 may serve as a cathode, configured to electrically contact cardiac tissue and deliver pacing pulses thereto. Pacing device 12 may be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes may be configured to electrically contact and deliver pacing pulses to cardiac tissue of different heart chambers. For example, one cathode electrode may be configured to electrically contact and deliver pacing pulses to atrial tissue, and another cathode electrode may be configured to electrically contact and deliver pacing pulses to ventricular tissue.

In the example of FIG. 2, housing 150 includes a first portion 152A and a second portion 152B. Portion 152B may, in some examples, define at least part of a power source case that houses a power source (e.g., a battery) of pacing device 12. The power source case may house a power source (e.g., a battery) of pacing device 12. In some examples, the portion 152B may include the conductive portion of housing that forms electrode 160.

Electrodes 100 and 160 are electrically isolated from each other. Electrode 100 may be referred to as a tip electrode, and fixation tines 110 may be configured to anchor pacing device 12 to cardiac tissue such that electrode 100 maintains contact with the cardiac tissue. In some examples, a portion of housing 150 may be covered by, or formed from, an insulative material to isolate electrodes 100 and 160 from each other and/or to provide a desired size and shape for one or both of electrodes 100 and 160. Electrode 160 may be a portion of housing 150, e.g., housing portion 152B, that does not include such insulative material. Electrode 160 can be most or all of housing 150, but most of housing 150 (other than electrode 160, may be covered with an insulative coating. Additionally or alternatively, electrode 160 may be coated with materials to promote conduction. In some examples, electrode 160 may be part of a separate ring portion of housing 150 that is conductive. Electrodes 100 and 160, which may include conductive portion(s) of housing 16, may be electrically connected to at least some electronics of pacing device 12 (e.g., sensing circuitry, electrical stimulation circuitry, or both). In some examples, housing 150 may include an end cap 172, which may include a feedthrough assembly to electrically couple electrode 100 to the electronics within housing 150, while electrically isolating electrode 100 from housing 150, e.g., including electrode 160 or other conductive portions of housing 150.

In the example of FIG. 2, the proximal end of pacing device 12 includes a flange 158 that defines an opening. Flange 158 may enable medical instruments to attach to pacing device 12, e.g., for delivery and/or extraction of pacing device 12. For example, a tether that extends through a catheter inserted into heart 16 (FIG. 1) may be attached to flange 158 and/or threaded through the opening to implant or extract pacing device 12.

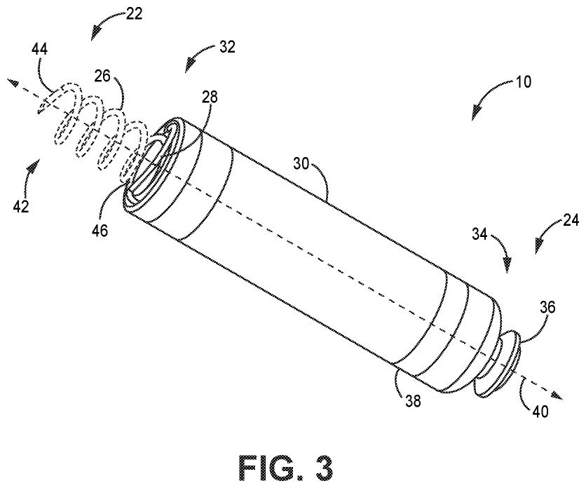

FIG. 3 is a perspective drawing illustrating an example of a pacing device 10 to sense in and/or deliver cardiac pacing to more than one chamber of a heart. Device 10 may be implanted in the right atrium (RA) of the patient's heart in a target implant region, such as the triangle of Koch, in the heart of the patient with a distal end of device 10 directed toward the left ventricle (LV) of the patient's heart. While the distal end of device 10 may be directed toward the LV, the distal end may be directed to other targets, such as interventricular septum of heart, in some examples.

Device 10 includes a housing 30 that defines a hermetically sealed internal cavity. Housing 30 extends between distal end 32 and proximal end 34. In some examples, housing can be cylindrical or substantially cylindrical but may be other shapes, e.g., prismatic or other geometric shapes. Housing 30 may include a delivery tool interface member 36, e.g., at proximal end 24, for engaging with a delivery tool during implantation of device 10.

All, substantially all, or a portion of housing 30 may function as an electrode 38, e.g., an anode, during pacing and/or sensing. In some examples, electrode 38 can circumscribe a portion of housing 30 at or near proximal end 34. Electrode 38 can fully or partially circumscribe housing 30. FIG. 3 shows electrode 38 extending as a singular band. Electrode 38 can also include multiple segments spaced a distance apart along a longitudinal axis 40 of housing 30 and/or around a perimeter of housing 30.

In some examples, electrode 38 may be a component, such as a ring electrode, that is mounted or assembled onto housing 30. Electrode 38 may be electrically coupled to internal circuitry of device 10 via electrically-conductive housing 30 or an electrical conductor when housing 30 is a non-conductive material. In some examples, electrode 38 is located proximate to proximal end 24 of housing 30 and can be referred to as a proximal housing-based electrode. Electrode 38 can also be located at other positions along housing 30, e.g., located proximately to distal end 22 or at other positions along longitudinal axis 40.

Each of first electrode 26 and second electrode 28 extends from a first end that is fixedly attached to housing 30 at or near distal end 22, to a second end that, in the example of FIG. 3, is not attached to housing 30 other than via the first end (e.g., is a free end). First electrode 26 includes one or more coatings configured to define a first electrically active region 44 and second electrode 28 includes one or more coatings configured to define a second electrically active region 46. In some examples, first electrically active region 44 can be more proximate to the second, e.g., distal, end of first electrode 26 than second electrically active region 46 is proximate to either end of second electrode 28. In the example of FIG. 3, first electrically active region 44 includes the distal end of electrode 26.

In the example of FIG. 3, first electrode 26 takes the form of a helix. In some examples, a helix is an object having a three-dimensional shape like that of a wire wound uniformly in a single layer around a cylindrical or conical surface such that the wire would be in a straight line if the surface were unrolled into a plane. Second electrode 28 includes a ramp portion 29, which may be configured as a partial helix, e.g., a helix that does not make a full revolution around a circumference of the cylindrical or conical surface.

As illustrated in FIG. 3, first electrode 26 may be a right-hand wound helix, and second electrode 28 may be a left-hand wound partial helix, although in other examples the handedness of the electrodes may be switched or the electrodes may have the same handedness as each other. In the example of FIG. 3, the helix and partial helix defined by first electrode 26 and second electrode 28, respectively, have the same pitch, although they may have different pitches in other examples. In some examples, one or both of electrodes 26 and 28 may have a shape other than helical. For example, the second electrode may have a loop shape in some examples. As another example, a first electrode configured to penetrate tissue of another chamber may be configured as one or more elongate darts, barbs, or tines.

First and second electrodes 26 and 28 can also vary in size and shape in order to enhance tissue contact of first and second electrically active regions 44 and 46. For example, first and second electrodes 26 and 28 can have a round cross section or could be made with a flatter cross section (e.g., oval or rectangular) based on tissue contact specifications.

The distal end of first electrode 26 can have a conical, hemi-spherical, or slanted edge distal tip with a narrow tip diameter, e.g., less than 1 millimeter (mm), for penetrating into and through tissue layers.

The outer dimensions of first electrode 26 may be substantially straight and cylindrical, with first electrode 26 being rigid in some examples. In some examples, first and second electrodes 26 and 28 can have flexibility in lateral directions, being non-rigid to allow some flexing with heart motion. In a relaxed state, when not subjected to any external forces, first and second electrodes 26 and 28 may be configured to maintain a distance between first and second electrically active regions 44 and 46 and housing distal end 32.

The configurations of first and second electrodes 26 and 28 illustrated in FIG. 3 are merely examples. In some examples, first electrode 26 may comprise one or more darts, tines, or other structures. In some examples, second electrode 28 may comprise one or more helices, darts, tines, buttons, pads, or other structures.

In some examples, second electrode 28 or electrode 38 may be paired with first electrode 26 for sensing ventricular signals and delivering ventricular pacing pulses. In some examples, second electrode 28 may be paired with electrode 38 or first electrode 26 for sensing atrial signals and delivering pacing pulses to atrial myocardium 20 in target implant region 2. In other words, electrode 38 may be paired, at different times, with both first electrode 26 and second electrode 28 for either ventricular or atrial functionality, respectively, in some examples. In some examples, first and second electrodes 26 and 28 may be paired with each other, with different polarities, for atrial and ventricular functionality.

In some examples, second electrode 28 may be configured as an atrial cathode electrode for delivering pacing pulses to the atrial tissue at target implant region in combination with electrode 38. Second electrode 28 and electrode 38 may also be used to sense atrial P-waves for use in controlling atrial pacing pulses (delivered in the absence of a sensed P-wave) and for controlling atrial-synchronized ventricular pacing pulses delivered using first electrode 26 as a cathode and electrode 38 as the return anode.

At distal end 22, device 10 includes a distal fixation assembly 42 including first electrode 26, second electrode 28, and housing distal end 32. A distal end of first electrode 26 can be configured to rest within a ventricular myocardium of the patient, and second electrode 28 can be configured to contact an atrial endocardium of the patient. In some examples, distal fixation assembly 42 can include more or less electrodes than two electrodes. In some examples, distal fixation assembly 42 may include one or more second electrodes along housing distal end 32. For example, distal fixation assembly 42 may include three electrodes configured for atrial functionality like second electrode 28, and the three electrodes may be substantially similar or different from one another. Spacing between a plurality of second electrodes 28 may be at an equal or unequal distance. Second electrode(s) 28 may be individually selectively coupled to sensing and/or pacing circuitry enclosed by housing 30 for use as an anode with first electrode 26 or as an atrial cathode electrode, or may be electrically common and not individually selectable.

It should be understood that, notwithstanding the specific examples of implantable medical devices and pacing devices disclosed herein, such as pacing device 10 and pacing device 12, the techniques disclosed herein for, inter alia, detection of HF may be implemented in any suitable implantable medical device or pacing device.

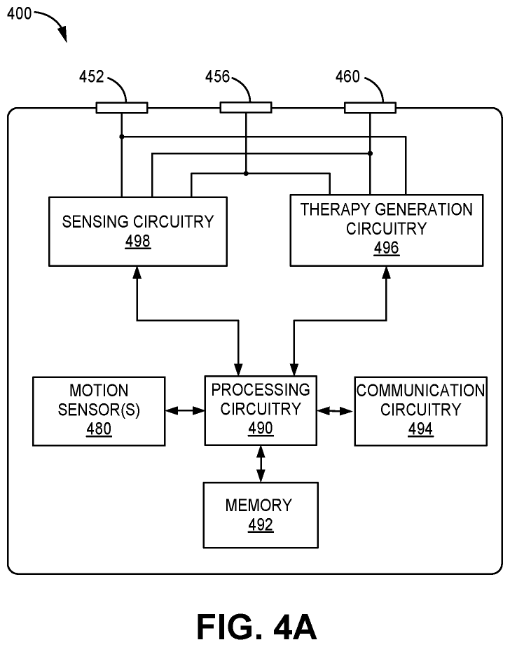

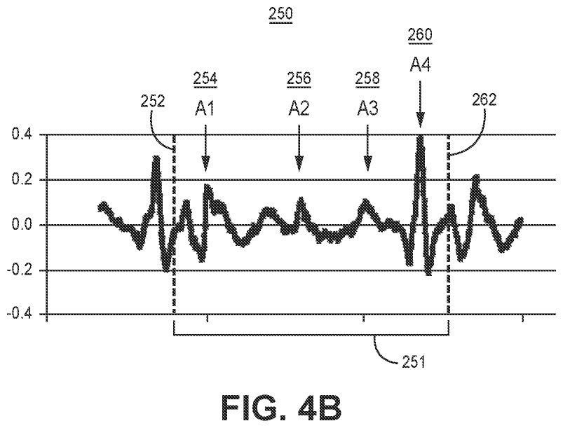

FIG. 4A is a conceptual block diagram of an example implantable medical device 400, in accordance with one or more aspects of this disclosure. In some examples, implantable medical device 400 may represent an example of pacing device 12, as shown in FIG. 2, or pacing device 10, as shown in FIG. 3. FIG. 4A shows an example of implantable medical device 400 having three electrodes, FIG. 2 shows an example of pacing device 12 having two electrodes, and FIG. 3 shows an example of pacing device 10 having three electrodes. However, the number of electrodes illustrated in FIGS. 2-4 are examples, and other numbers of electrodes may be included in implantable medical device 400, pacing device 12, or pacing device 10, such as, but not limited to, 2-10 electrodes. In some examples, the number of electrodes included in implantable medical device 400, pacing device 12, or pacing device 10 may be more than 10 electrodes.

In the illustrated example, implantable medical device 400 may include one or more of processing circuitry 490, memory 492, therapy generation circuitry 496, sensing circuitry 498, motion sensor 480, and/or communication circuitry 494. One or more of the elements of implantable medical device 400 may be part of an electronics module. For example, processing circuitry 490, memory 492, therapy generation circuitry 496, sensing circuitry 498, motion sensor 480, and/or communication circuitry 494 may be mounted on a circuit board of an electronics module of implantable medical device 400.

Memory 492 may include computer-readable instructions that, when executed by processing circuitry 490, cause implantable medical device 400 and processing circuitry 490 to perform various functions of implantable medical device 400 such as storing and analyzing signals received by implantable medical device 400 and providing pacing therapy for a patient's heart.

Memory 492 may include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

Processing circuitry 490 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 490 may include multiple components, such as any combination of one or more microprocessors, one or more controllers, one or more DSPs, one or more ASICs, or one or more FPGAs, as well as other discrete or integrated logic circuitry. The functions attributed to processing circuitry 490 herein may be embodied as software, firmware, hardware or any combination thereof.

Processing circuitry 490 may control therapy generation circuitry 496 to deliver stimulation therapy to a patient's heart according to therapy parameters, which may be stored in memory 492. For example, processing circuitry 490 may control therapy generation circuitry 496 to deliver electrical pulses with the amplitudes, pulse widths, frequency, or electrode polarities specified by the therapy parameters. In this manner, therapy generation circuitry 496 may deliver pacing pulses to the heart via electrodes 452, 456, and/or 460.

Although implantable medical device 400 may only include two electrodes, e.g., electrodes 452 and 460, implantable medical device 400 may utilize three or more electrodes in other examples. Implantable medical device 400 may use any combination of electrodes to deliver therapy and/or detect electrical signals from the patient.

Therapy generation circuitry 496 may be electrically coupled to electrodes 452, 456, and/or 460 positioned on the housing of implantable medical device 400. In the illustrated example, therapy generation circuitry 496 is configured to generate and deliver electrical stimulation therapy to the heart. For example, therapy generation circuitry 496 may deliver pulses to a portion of cardiac muscle within the heart via electrodes 452, 456, and/or 460. In some examples, therapy generation circuitry 496 may deliver pacing stimulation in the form of electrical pulses. Therapy generation circuitry 496 may include charging circuitry, and one or more charge storage devices, such as one or more capacitors. Switching circuitry (not shown) may control when the capacitor(s) are discharged to electrodes 452 and 460.

Sensing circuitry 498 may monitor signals from at least one of electrodes 452, 456, and 460 to monitor electrical activity of the heart, impedance, or another electrical phenomenon. Sensing may be done to determine heart rates or heart rate variability, or to detect ventricular dyssynchrony, arrhythmias (e.g., tachyarrhythmias) or other electrical signals. Sensing circuitry 498 may include switching circuitry to select the electrode polarity used to sense the heart activity. In examples with more than two electrodes, processing circuitry 490 may select the electrodes that function as sense electrodes, i.e., select the sensing configuration, via the switching circuitry within sensing circuitry 498. In some examples, electrode 452 is connected to a first pole of a battery of implantable medical device 400 (e.g., the positive terminal of the battery), electrode 460 is connected to a second pole of the battery (e.g., the case ground), and electrode 456 is a sense electrode configured to receive signals in the environment surrounding implantable medical device 400. Other configurations of electrodes 452, 456, and 460 are also possible.

Motion sensor 480 may be contained within the housing of implantable medical device 400 and include one or more accelerometers, gyroscopes, electrical or magnetic field sensors, or other devices capable of detecting motion and/or position of implantable medical device 400. For example, motion sensor 480 may include a three-axis accelerometer (three-dimensional accelerometer) that is configured to detect accelerations in any direction in space. Specifically, the three-axis accelerometer may be used to detect the motion of implantable medical device 400 that may be indicative of cardiac events and/or noise. In some examples, motion sensor 480 may include a 6-axis accelerometer. In some examples, motion sensor 480 may include a 9-axis accelerometer. The motion sensor(s) 480 may be sensitive to the motion of the heart 16, including the paced activation of the ventricles.

While processing circuitry 490 controls therapy generation circuitry 496 to deliver ventricular pacing pulses, processing circuitry 490 may also control or monitor motion sensor(s) 480 to generate a signal that varies with the cardiac contraction. In some examples, motion sensor(s) 480 may generate the signal substantially continuously. Processing circuitry 490 may identify one or more features of the cardiac contraction within the signal, on a beat-by-beat basis, or otherwise, to facilitate, e.g., delivery of ventricular pacing pulses in an atrial-synchronized manner.

FIG. 4B is an example of a motion sensor signal 250 that may be acquired by motion sensor(s) 480 over a cardiac cycle. Vertical dashed lines 252 and 262 denote the timing of two consecutive ventricular events (an intrinsic ventricular depolarization or a ventricular pace), marking the respective beginning and end of the ventricular cycle 251. The motion signal includes an A1 event 254, an A2 event 256, an A3 event 258 and an A4 event 260. The A1 event 254 is an acceleration signal (in this example when motion sensor(s) 480 is/are implemented as one or more accelerometers) that occurs during ventricular contraction and marks the approximate onset of ventricular mechanical systole. The A1 event, which may correspond roughly to the S1 heart sound, is also referred to herein as a “ventricular contraction event.” The A2 event 265 is an acceleration signal that occurs during ventricular relaxation and marks the approximate offset or end of ventricular mechanical systole. The A2 event, which may correspond roughly to the S2 heart sound, is also referred to herein as the “ventricular relaxation event.” The A3 event 258 is an acceleration signal that occurs during passive ventricular filling and marks ventricular mechanical diastole. The A3 event, which may correspond roughly to the S3 heart sound, is also referred to herein as the “ventricular passive filling event.” Since the A2 event occurs with the end of ventricular systole, it is an indicator of the onset of ventricular diastole. The A3 event occurs during ventricular diastole. As such, the A2 and A3 events may be collectively referred to as ventricular mechanical diastolic events because they are both indicators of the ventricular diastolic period.

The A4 event 260 is an acceleration signal that occurs during atrial contraction and active ventricular filling and marks atrial mechanical systole. The A4 event 260 is also referred to herein as the “atrial systolic event” or merely the “atrial event,” and is the atrial systolic event that may be detected from motion sensor signal 250 to trigger ventricular pacing pulse delivery by starting an AV interval or AV delay in response to detecting the A4 event 260. Processing circuitry 490 and/or other components of implantable medical device 400 may be configured to detect one or more of the A1, A2, A3, and A4 events from motion sensor signal 250, for some or all cardiac cycles during which such functionality is enabled.

Communication circuitry 494 includes any suitable hardware, firmware, software or any combination thereof for communicating with another device, such as an external device or another implantable device. In some examples, communication circuitry 494 may be configured for tissue conductive communication with another implantable medical device via electrodes 452, 456, and/or 460. Implantable medical device 400 may communicate with an external device via the other implantable medical device, or communication circuitry 494 may be configured for radio-frequency communication with an external device, e.g., via an antenna.

Implantable medical device 400 may deliver an electrical stimulation therapy pacing protocol to heart 16 via one or more of the plurality of electrodes 452, 456, and 460 over a period of time to increase a heartrate of a heart 16 of patient 14 to at least a target rate for cardiac motion monitoring during the period of time. For example, the period of time may be greater than or equal to 10 seconds and less than or equal to 30 seconds, such as for 10 seconds, 15 seconds, 20 seconds, 25, seconds, or 30 seconds. In some examples, the period of time may be greater than 30 seconds or less than 10 seconds. Examples of a target rate may be within a range from 100 beats per minute (BPM) to 150 BPM. For example, the target rate may be set at 120 BPM, 110 BPM, 130 BPM, etc. In some examples, the target rate may be lower than 100 BPM or over 150 BPM. In some examples, the target rate may be set by setting an interval that controls when a pacing pulse is delivered after a preceding paced or intrinsic depolarization. In some examples, the target rate may be a percent increase of the patient's resting heartrate, such as 110%, 120%, 125%, 135%, or 150% of the patient's resting heartrate.

In some examples, sensing circuitry 498 may be configured to detect events, e.g., depolarizations, within the cardiac electrical signals, and provide indications thereof to processing circuitry 490. For example, sensing circuitry 498 may detect the events via motion sensor(s) 480, such as one or more accelerometers, and/or via one or more electrodes 452, 456, 460, sensing intrinsic or evoked cardiac electrical signals. In this manner, processing circuitry 490 may be configured to determine the timing of atrial and/or ventricular depolarizations or contractions, and control the delivery of cardiac pacing, e.g., atrioventricular (AV) synchronized cardiac pacing, based thereon. In some examples, processing circuitry 490 of implantable medical device 400 may additionally or alternatively determine heartrate based on sensed (intrinsic) depolarizations, determine whether the heartrate is at or above the target rate, and then collect motion data from motion sensor(s) to determine an indication of HF.

In some examples, implantable medical device 400 may detect atrial and/or ventricular depolarizations or atrial and/or ventricular contractions via motion sensor(s) 480, such as an accelerometer and deliver ventricular pacing pulses after an AV interval or AV delay, The AV interval/delay is a time between the detection of atrial depolarization or contraction and the delivery of ventricular pacing pulses. In some examples, processing circuitry 490 may perform an AV interval sweep to deliver pacing pulses at a variety of AV intervals over a period of time or over a number of cardiac cycles. Processing circuitry 490 may perform such an AV interval sweep independently or in combination with pacing the heart at one or more elevated rates, e.g. according to a predefined pacing challenge protocol, including any such protocol described herein, or otherwise.

Motion sensor(s) 480 included in implantable medical device 400 may detect motion of the heart, such as during the period of time a pacing protocol is delivered to heart 16 to temporarily increase the heartrate. Implantable medical device 400 may determine an indication of HF based on the detected motion of implantable device 400 during the period of time a pacing protocol is delivered to heart 16 to temporarily increase the heartrate. For example, implantable medical device 400 may determine one or more features of heart motion (“FHMs”) of heart 16 based on the detected motion. Some examples of FHMs may include one or more of amplitude of the A1 event (or the S1 heart sound), rate of A1 event (or S1 heart sound) onset, duration of the A1 event, A1 to A3 (or S1 to S3 heart sound) interval, rate of A3 event onset, amplitude of the A3 event, or duration of the A3 event. Implantable medical device 400 may determine a difference between the one or more determined FHMs and one or more respective FHM threshold(s) and determine an indication of HF based on the determined difference.

In some examples, the FHM threshold(s) may be determined during an initial period of time after implantable medical device 400 is implanted, to serve as a baseline of cardiac function for patient 14. For example, implantable medical device 400 may determine one or more FHMs based on detected heart motion during pacing protocols performed during an initial period of time, such as the first week, two weeks, month, two months, etc. after implantable medical device 400 is implanted. In some examples, an average, median, mode, etc. of FHM values compiled during the initial period of time may then be used to determine the FHM threshold(s). Afterwards, implantable medical device 400 may perform one or more pacing challenge protocols, which may involve pacing the heart at one or more elevated rates, and/or performing a simultaneous (or prior or subsequent) AV interval sweep. In the process, implantable medical device 400 may measure one or more FHMs, such as any of the various FHMs described herein, or any other FHM(s). Implantable medical device 400 may then determine any difference(s) between the measured one or more FHMs and the respective FHM threshold(s), and determine an indication of HF based on any determined difference(s). In this manner, implantable medical device 400 may determine whether the patient may be afflicted with HF, the apparent severity of any HF, and/or whether any previously detected HF in patient 14 is changing, e.g., getting worse. In some examples, an FHM threshold may be predetermined, such as corresponding to a known value that indicates normal cardiac function for most patients, or for a typical patient, or for a comparable patient.

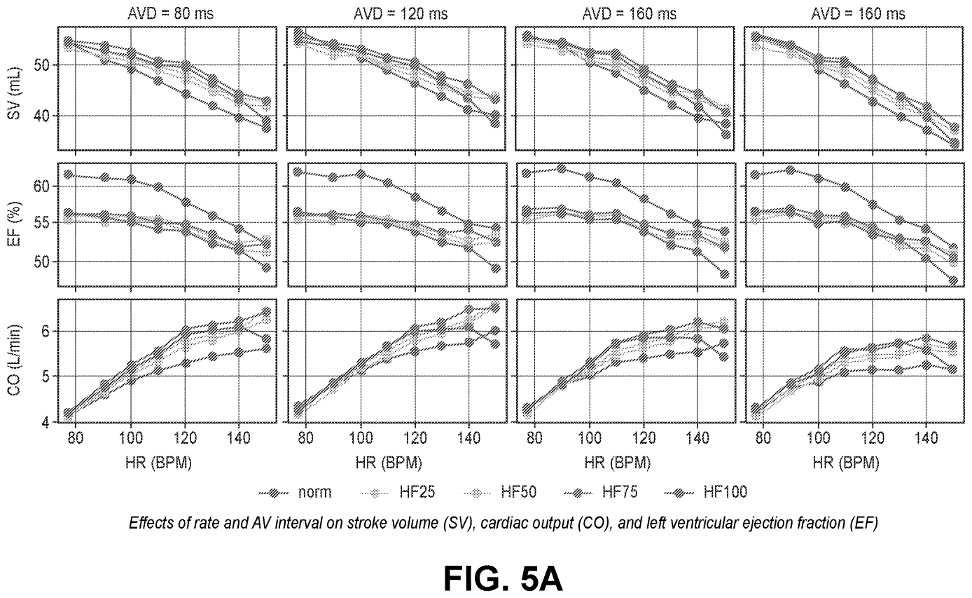

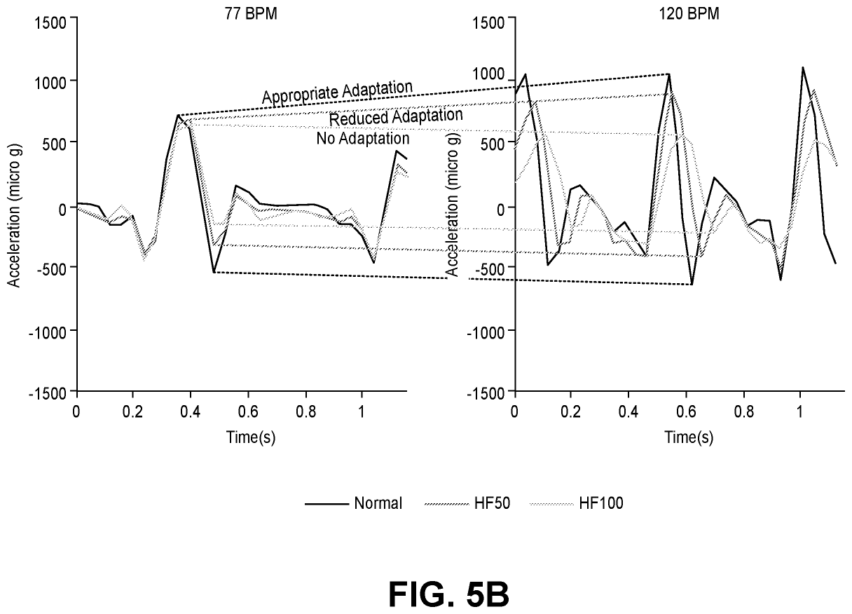

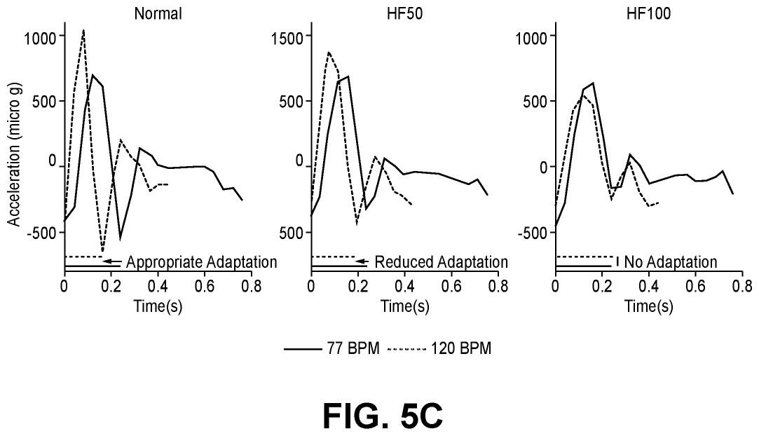

FIG. 5A provides a series of plots of stroke volume (SV), ejection fraction (EF) and cardiac output (CO) for five computer-modeled, simulated hearts: a heart with normal cardiac function (“norm”), and hearts with increasing degrees of HF (“HF25” (least severe) “HF100” (most severe)). The plots are provided for a range of four different atrioventricular delay intervals (“AVD”) from 80 ms to 200 ms, and each plot depicts the labeled measure for all five hearts across a range of heartrates from just below 80 BPM to above 140 BPM. FIG. 5A reveals that HF may be masked at resting rates in some measures of cardiac function, but become more pronounced at increased heartrates. For example, stroke volume and cardiac output show little to no difference between a heart with normal cardiac function and a heart with HF when the heart is at a resting heartrate. However, as heartrates increase above a resting rate, differences between a heart with normal cardiac function and a heart afflicted by HF may become more pronounced.

Implantable medical device 400 may challenge the heart 16 of a patient 14 with high-rate pacing during a period of time, or over a number of cardiac cycles, to elicit differentiating changes in the mechanical response measured by motion sensor(s) 480 of implantable medical device 400. In some examples, implantable medical device 400 may challenge the heart 16 with high-rate pacing and/or simultaneous or non-simultaneous AV interval sweeps during a period of time or over a number of cardiac cycles to elicit differentiating changes in the mechanical response measured by motion sensor(s) 480 of implantable medical device 400. In some examples, a train of pacing to elicit a desired response may include 10 to 20 beats, which may correspond to the period of time being greater than or equal to 10 seconds and less than or equal to 30 seconds, such as for 10 seconds, 15 seconds, 20 seconds, 25, seconds, or 30 seconds. In some examples, the period of time may be greater than 30 seconds or less than 10 seconds. Processing circuitry 490 of implantable medical device 400 may determine an indication of HF based on the measurements by motion sensor(s) 480.

Implantable medical device 400 may create and/or execute a virtual stress test through a pacing rate protocol to detect dysfunction in the heart 16 of a patient 14 by sensing signs of such dysfunction mechanically via motion sensor(s) 480 of implantable medical device 400. In this manner, implantable medical device 400 may provide effective detection of patients progressing into HF.

In some examples, cardiac function corresponds to a level of HF. For example, significantly impaired cardiac function may correspond to a high level of HF. Normal cardiac function may correspond to a heart that may have little to no HF. Differences between one or more measurements of motion sensor(s) 480 of normal cardiac function, moderately impaired cardiac function and significantly impaired cardiac function may become more pronounced when heartrate is increased above a resting heartrate and/or above a heartrate pacing threshold.

FIG. 5B depicts the acceleration, during cardiac cycles occurring over a 1.1-1.2 second span of time, of heart tissue at a right ventricular location in a simulated dilated. cardiomyopathic human heart (four locations along the right ventricular septal joint, and one in the triangle of Koch). On the left side of FIG. 5B, the measured acceleration is depicted at a simulated heartrate of 77 BPM, and on the right side the measured acceleration is depicted at a simulated heartrate of 120 BPM. On each side, acceleration curves are plotted for the heart with normal cardiac function (“Normal”), the heart with moderate HF (“HF50”), and the heart with severe HF (“HF100”). A primary change in the simulated heart model among the normal, moderate HF and severe HF states is the contractility of the myocardium. As shown in FIG. 5B, when heartrate is increased from a resting heartrate, such as 77 BPM, to or above a pacing threshold, such as 120 BPM, the amplitude of the acceleration at the time of the S1 heart sound (and/or elsewhere in the cardiac cycle) may increase more in a heart with normal cardiac function than in a heart with moderate HF or severe HF. More specifically, the normal heart showed the greatest increase in acceleration at the S1 heart sound, the heart with moderate HF showed a lower increase in acceleration at S1, and the heart with severe HF showed the lowest increase in acceleration at S1 of all three. In some examples, the FHM threshold may comprise an increase in acceleration amplitude at S1 for a heart with normal cardiac function (when paced at a relatively high rate). In some examples, the FHM threshold may comprise an increase in acceleration amplitude at S1 for heart 16 of patient 14 when paced at a relatively high rate during the initial time period after implantable medical device 400 is implanted. In some examples, the more the cardiac function of a heart is impaired (e.g., due to HF), the less the acceleration amplitude at A1 will increase when heartrate is increased to or above a pacing threshold. In some examples, the FHM(s) may include one or more of rate of A1 onset, acceleration amplitude at A1, duration of A1, A1 to A3 interval, rate of A3 onset, acceleration amplitude at A3, or duration of A3. In some examples, the FHM threshold(s) may include an increase in any one or more of the following, when paced at a relatively high rate: rate of A1 onset, acceleration amplitude at A1, duration of A1, A1 to A3 interval, rate of A3 onset, acceleration amplitude at A3, or duration of A3 for a heart with normal cardiac function or for the heart 16 of patient 14 during the initial time period after implantable medical device 400 is implanted.

FIG. 5C depicts the duration of the S1 event as observed in the acceleration data compiled for FIG. 5B, with respect to the same three simulated hearts (Normal, HF50, HF100). Each plot provides a comparison for an individual heart between S1 duration at 77 BPM and S1 duration at 120 BPM. In the heart with normal function, the duration of the S1 signal decreases when heartrate is increased. The hearts with HF show the amount of decrease in duration of S1 is less when heartrate is increased as a level of HF increases. For example, the decrease in S1 duration of the HF50 heart in response to a higher heartrate is reduced compared to a heart with normal function. The decrease in S1 duration of the HF100 heart in response to higher heartrate is at or close to zero, and the lowest of all three hearts. Accordingly, the decrease in S1 duration of the HF100 heart in response to higher heartrate is significantly reduced compared to a heart with normal function since there is little to no change in S1 duration of the HF100 heart when heartrate is increased.

FIGS. 5B-SC show that heartrate acts to significantly differentiate normal and HF hearts. The amplitude (FIG. 5B) and duration (FIG. 5C) of the acceleration signal generated by the motion sensor(s) 480 associated with systolic contraction shows adaption to the increased heartrate by a heart with normal function, i.e., increasing amplitude and shortening duration. However, this adaption is either blunted, or abolished in the hearts with HF (HF50 and HF 100). For example, in FIG. 5B, the amplitude of the S1 signal in the heart with normal function increases when heartrate is increased. The HF hearts show reduction or disappearance of this increase in amplitude of the S1 signal when heartrate is increased.

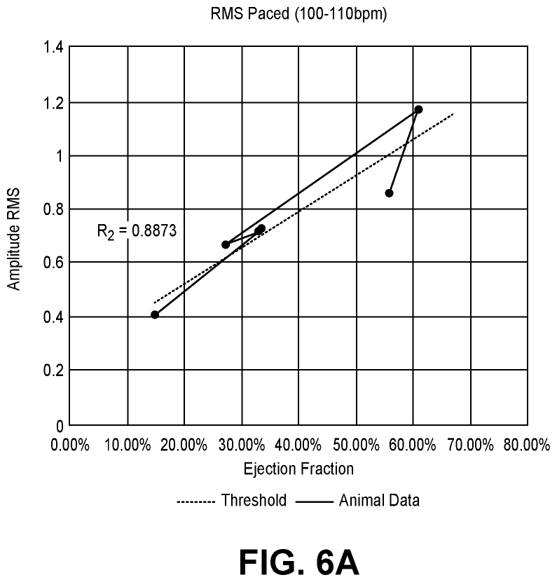

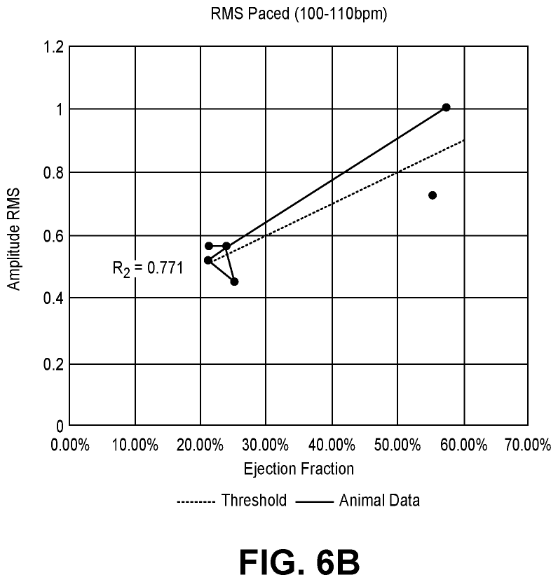

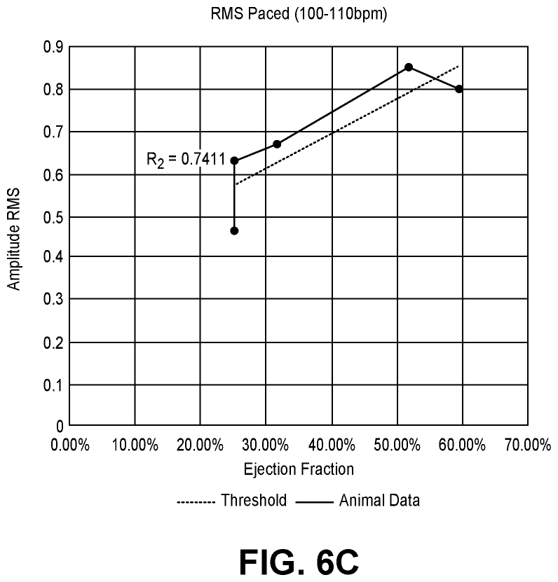

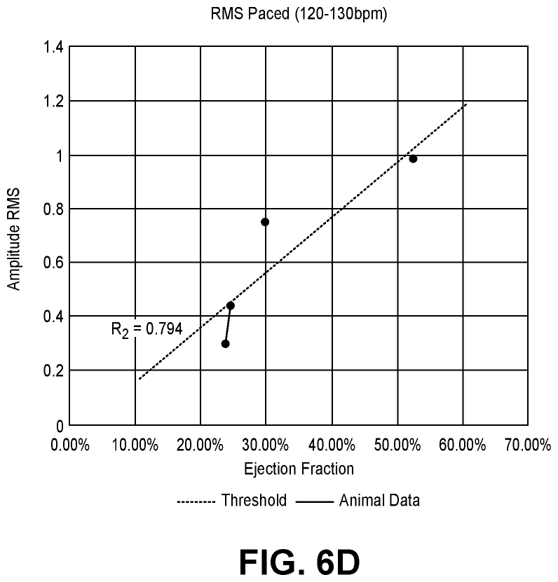

In some examples, a mean motion sensor amplitude, such as a root mean square (RMS) amplitude or a mean of a rectified amplitude waveform or plot, may be employed to determine an indication of HF. FIGS. 6A-6D depict plots of motion sensor amplitude RMS recorded with leadless intracardiac pacemakers (Micra™ AV from Medtronic, Inc.) implanted in the right ventricles of several study animals. For a period of time prior to data recordation, the animals were paced at an accelerated rate in order to induce conditions resembling HF, including declining ejection fraction (EF). Motion sensor (accelerometer) amplitude data was then collected from the pacemakers during periods of pacing at an elevated heart rate (above resting heartrate, typically in a range of 100-110 BPM or 120-130 BPM). EF was measured simultaneously via ultrasound. The plots of FIGS. 6A-6D show a strong correlation between amplitude RMS and EF: as EF declines, so does amplitude RMS. Consequently, motion sensor amplitude RMS can be employed to detect an indication of HF. For example, a downward trend in motion sensor amplitude RMS, or a motion sensor amplitude RMS falling below a threshold value, can be employed to detect an indication of HF. Motion sensor amplitude RMS data can be collected from a given patient in a single pacing session, or multiple pacing sessions with a time gap (e.g., one or more minutes, hours, days, weeks, months) between the pacing sessions. Such data can be collected at an elevated target heart rate as discussed above, or at a non-elevated target heart rate, and/or at multiple target heart rates. Where such data is collected in multiple pacing sessions, the same target heartrate or set of target heartrates may be employed in some or all of the pacing sessions to facilitate comparison of the motion sensor amplitude RMS (or any other mean motion sensor amplitude) data collected in the sessions and a determination of an indication of HF based on the collected data, or a trend in the collected data among the pacing sessions.

The techniques described herein may facilitate detection of an indication of HF, which may include detecting an indication of HF before clinical symptoms emerge, or before clinical symptoms require intervention. An indication of HF may include, without limitation, any one or more of: a presence or absence of HF, a possibility of HF or of one or more symptoms of HF, an onset of HF, a form of HF, a type of HF, an extent of HF, a severity of HF, a level of HF, or a change in HF severity and/or form. Testing for HF by implantable medical device 400 using any of the techniques disclosed herein may occur between physician office visits by a patient, which may provide an earlier indication of HF to physicians. These early indications would allow for earlier interventions which would result in better clinical outcomes for these patients. In some examples, implantable medical device 400 may also provide feedback that clinical interventions are providing benefits to the patient and/or enhance patient compliance with clinical interventions. In addition, implantable medical device 400 may execute any of the pacing challenge protocols and/or HF assessment techniques described herein concurrently with its execution of pacing capture management protocols, which may result beneficially result in little to no additional risk to patients, as such a practice would reduce or minimize any added non-demand high-rate pacing to be performed on the patient.

In some examples, sensing circuitry 498 may be configured to monitor signals from one or more of the plurality of electrodes 452, 456, 460 to monitor one or more of electrical activity of the heart 16, impedance, or other electrical phenomenon. Processing circuitry 490 of implantable medical device 400 may be configured to determine an indication of HF based on the motion detected by motion sensor(s) 480 during the appropriate period(s) of time, and/or on any other monitored signals.

In some examples, the FHM threshold may comprise an amount of decrease in duration of the S1 signal, when paced at a relatively high rate, for a heart with normal cardiac function or for heart 16 of patient 14 during the initial time period after implantable medical device 400 is implanted. A subsequently measured amount of decrease in duration of the S1 signal of heart 16 may be compared to the FHM threshold to determine an indication of HF.

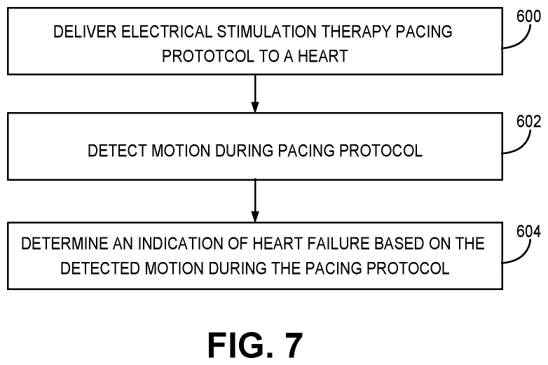

FIG. 7 is a flow diagram illustrating an example process that may be executed by implantable medical device 400 or any other suitable implantable medical device, in accordance with one or more aspects of this disclosure. The techniques of FIG. 7 are described with reference to implantable medical device 400 shown in FIG. 4, although other components may exemplify similar techniques.

In the example of FIG. 7, implantable medical device 400 may deliver an electrical stimulation therapy pacing protocol to heart 16 of patient 14 (600), via one or more of a plurality of electrodes 452, 456, 460, according to any technique disclosed herein, or otherwise. Implantable medical device 400 may detect motion during the pacing protocol (602), such as via motion sensor(s) 480 included in implantable medical device 400. Implantable medical device 400 may determine an indication of HF based on the detected motion during pacing protocol (604).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes a plurality of electrodes; a motion sensor configured to detect motion; therapy generation circuitry electrically coupled to one or more of the plurality of electrodes; and processing circuitry configured to: control the therapy generation circuitry to deliver an electrical stimulation therapy pacing protocol to a heart via one or more of the plurality of electrodes over a period of time to increase heartrate during the period of time to at least a target heartrate; and determine an indication of heart failure based on the detected motion during the period of time.

Example 2: The system of example 1, wherein the indication of heart failure is one or more of an extent of heart failure, a form of heart failure, a presence or absence of heart failure, a possibility of heart failure, or a change in heart failure severity.

Example 3: The system of any of examples 1-2, wherein to determine an indication of heart failure based on the detected motion during the period of time includes: determine a feature of heart motion of the heart based on the detected motion; determine a difference between the determined feature of heart motion and a respective feature of heart motion threshold; and determine the indication of heart failure based on the determined difference.

Example 4: The system of any of examples 1-2, wherein to determine an indication of heart failure based on the detected motion during the period of time includes: determine a change in value of a feature of heart motion of the heart based on the detected motion; determine a difference between the determined change in value of a feature of heart motion and a respective change threshold; and determine the indication of heart failure based on the determined difference.

Example 5: The system of example 4, wherein the change in value is between the feature of heart motion at a resting rate and the feature of heart motion at an elevated pacing rate.

Example 6: The system of any of examples 3-5, wherein the feature of heart motion is a mean motion sensor amplitude.

Example 7: The system of example 6, wherein the mean motion sensor amplitude is a root mean square amplitude.

Example 8: The system of any of examples 3-7, wherein the feature of heart motion is a mean motion sensor amplitude throughout the period of time.

Example 9: The system of any of examples 3-4, wherein the feature of heart motion includes a local peak amplitude of the motion sensor signal coincident with ventricular systole.

Example 10: The system of any of examples 3-4, wherein the feature of heart motion includes a local peak amplitude of the motion sensor signal coincident with an S1 heart sound.

Example 11: The system of any of examples 3-4, wherein the feature of heart motion includes a duration or rate of onset of a local peak amplitude of the motion sensor signal coincident with an S1 heart sound.

Example 12: The system of any of examples 3-4, wherein the feature of heart motion includes one or more of: rate of onset of a local peak amplitude of the motion sensor coincident with ventricular systole, or duration of the local peak amplitude of the motion sensor coincident with ventricular systole.

Example 13: The system of any of examples 3-4, wherein the feature of heart motion includes one or more of: an interval from a local peak amplitude of the motion sensor signal coincident with ventricular systole to a local peak amplitude of the motion sensor coincident with ventricular diastole, or rate of onset, duration or magnitude of the local peak amplitude of the motion sensor coincident with ventricular diastole.

Example 14: The system of any of examples 1-13, wherein the period of time is greater than or equal to 10 seconds and less than or equal to 30 seconds.

Example 15: The system of any of examples 1-14, further comprising sensing circuitry configured to monitor signals from one or more of the plurality of electrodes to monitor one or more of electrical activity of the heart, impedance, or another electrical phenomenon.

Example 16: The system of example 15, wherein the processing circuitry is configured to determine the indication of heart failure based on the detected motion during the period of time and the monitored signals.

Example 17: The system of any of examples 1-16, wherein the motion sensor is an accelerometer.

Example 18: The system of any of examples 1-17, wherein the accelerometer is a 3-axis accelerometer.

Example 19: The system of any of examples 1-17, wherein the accelerometer is a 6-axis accelerometer.

Example 20: The system of any of examples 1-17, wherein the accelerometer is a 9-axis accelerometer.

Example 21: The system of any of examples 1-20, wherein the target heart rate is greater than or equal to 100 beats per minute and less than or equal to 150 beats per minute.

Example 22: The system of any of examples 1-21, wherein the plurality of electrodes, the motion sensor, the therapy generation circuitry, and the processing circuitry are included in an implantable medical device.

Example 23: The system of example 22, wherein the implantable medical device further includes the sensing circuitry.

Example 24: The system of any of examples 1-23, further comprising telemetry circuitry configured to output information pertaining to the determined indication of heart failure to a second device or network.

Example 25: The system of any of examples 1-24, wherein the electrical stimulation therapy pacing protocol includes delivering pacing stimulation signals to the heart.

Example 26: The system of example 25, wherein the electrical stimulation therapy pacing protocol further includes delivering the pacing stimulation signals to the heart at a plurality of atrioventricular intervals.

Example 27: A system includes a plurality of electrodes; a motion sensor configured to detect motion; therapy generation circuitry electrically coupled to one or more of the plurality of electrodes; and processing circuitry configured to: determine, based on the detected motion, a heartrate of a heart is at least a target threshold; in response to determining the heartrate is at least the target threshold, control the therapy generation circuitry to deliver, via one or more of the plurality of electrodes, cardiac pacing to the heart at a plurality of atrioventricular intervals over a period of time; and determine an indication of heart failure based on the detected motion during the period of time.

Example 28: The system of example 27, wherein the indication of heart failure is one or more of an extent of heart failure, a form of heart failure, a presence or absence of heart failure, a possibility of heart failure, or a change in heart failure severity.

Example 29: The system of any of examples 27-28, wherein to determine an indication of heart failure based on the detected motion during the period of time includes: determine a feature of heart motion of the heart based on the detected motion during the period of time; determine a difference between the feature of heart motion and a respective feature of heart motion threshold; and determine the indication of heart failure based on the determined difference.

Example 30: The system of any of examples 27-28, wherein to determine an indication of heart failure based on the detected motion during the period of time includes: determine a change in value of a feature of heart motion of the heart based on the detected motion during the period of time; determine a difference between the determined change in value of a feature of heart motion and a respective change threshold; and determine the indication of heart failure based on the determined difference.

Example 31: The system of example 30, wherein the change in value is between the feature of heart motion at a resting rate and the feature of heart motion at an elevated pacing rate.

Example 32: The system of any of examples 29-30, wherein the feature of heart motion is a mean motion sensor amplitude.

Example 33: The system of example 32, wherein the mean motion sensor amplitude is a root mean square amplitude.

Example 34: The system of any of examples 29-33, wherein the feature of heart motion is a mean motion sensor amplitude throughout the period of time.

Example 35: The system of any of examples 29-30, wherein the feature of heart motion includes a local peak amplitude of the motion sensor signal coincident with ventricular systole.

Example 36: The system of any of examples 29-30, wherein the feature of heart motion includes a local peak amplitude of the motion sensor signal coincident with an S1 heart sound.

Example 37: The system of any of examples 29-30, wherein the feature of heart motion includes a duration or rate of onset of a local peak amplitude of the motion sensor signal coincident with an S1 heart sound.

Example 38: The system of any of examples 29-30, wherein the feature of heart motion includes one or more of: rate of onset of a local peak amplitude of the motion sensor coincident with ventricular systole, or duration of the local peak amplitude of the motion sensor coincident with ventricular systole.

Example 39: The system of any of examples 29-30, wherein the feature of heart motion includes one or more of: an interval from a local peak amplitude of the motion sensor signal coincident with ventricular systole to a local peak amplitude of the motion sensor coincident with ventricular diastole, or rate of onset, duration or magnitude of the local peak amplitude of the motion sensor coincident with ventricular diastole.

Example 40: The system of any of examples 27-39, wherein the target heart rate is greater than or equal to 100 beats per minute and less than or equal to 150 beats per minute.

Example 41: The system of any of examples 27-40, wherein the plurality of electrodes, the motion sensor, the therapy generation circuitry, and the processing circuitry are included in an implantable medical device.

Example 42: The system of any of examples 27-41, wherein the motion sensor is an accelerometer.

Example 43: The system of any of examples 27-42, further comprising sensing circuitry configured to monitor signals from one or more of the plurality of electrodes to monitor one or more of electrical activity of the heart, impedance, or another electrical phenomenon.

Example 44: The system of example 43, wherein the processing circuitry is configured to determine the indication of heart failure based on the detected motion during the period of time and the monitored signals.

Example 45: The system of any of examples 27-44, further comprising telemetry circuitry configured to output information pertaining to the determined indication of heart failure to a second device or network.

Example 46: The system of any of examples 27-45, wherein the electrical stimulation therapy pacing protocol includes delivering pacing stimulation signals to the heart.

Example 47: The system of example 46, wherein the electrical stimulation therapy pacing protocol further includes delivering the pacing stimulation signals to the heart at a plurality of atrioventricular intervals.

Example 48: A method including delivering, by circuitry and via one or more of a plurality of electrodes, an electrical stimulation therapy pacing protocol to a heart to increase heartrate during a period of time to at least a target heartrate; detecting, by a motion sensor, motion during the pacing protocol during the period of time; and determining, by the circuitry, an indication of heart failure of the heart based on the detected motion during the period of time.

Example 49: The method of example 48, wherein the indication of heart failure is one or more of an extent of heart failure, a form of heart failure, a presence or absence of heart failure, a possibility of heart failure, or a change in heart failure severity.

Example 50: The method of any of examples 48-49, wherein determining an indication of heart failure based on the detected motion during the period of time includes: determining a feature of heart motion of the heart based on the detected motion; determine a difference between the determined feature of heart motion and a respective feature of heart motion threshold; and determine the indication of heart failure based on the determined difference.

Example 51: The method of any of examples 48-49, wherein determining an indication of heart failure based on the detected motion during the period of time includes: determining a change in value of a feature of heart motion of the heart based on the detected motion; determining a difference between the determined change in value of a feature of heart motion and a respective change threshold; and determining the indication of heart failure based on the determined difference.

Example 52: The method of example 51, wherein the change in value is between the feature of heart motion at a resting rate and the feature of heart motion at an elevated pacing rate.

Example 53: The method of any of examples 50-52, wherein the feature of heart motion is a mean motion sensor amplitude.

Example 54: The method of example 53, wherein the mean motion sensor amplitude is a root mean square amplitude.

Example 55: The method of any of examples 50-54, wherein the feature of heart motion is a mean motion sensor amplitude throughout the period of time.

Example 56: The method of any of examples 50-51, wherein the feature of heart motion includes a local peak amplitude of the motion sensor signal coincident with ventricular systole.

Example 57: The method of any of examples 50-51, wherein the feature of heart motion includes a local peak amplitude of the motion sensor signal coincident with an S1 heart sound.

Example 58: The method of any of examples 50-51, wherein the feature of heart motion includes a duration or rate of onset of a local peak amplitude of the motion sensor signal coincident with an S1 heart sound.

Example 59: The method of any of examples 50-51, wherein the feature of heart motion includes one or more of: rate of onset of a local peak amplitude of the motion sensor coincident with ventricular systole, or duration of the local peak amplitude of the motion sensor coincident with ventricular systole.

Example 60: The method of any of examples 50-51, wherein the feature of heart motion includes one or more of: an interval from a local peak amplitude of the motion sensor signal coincident with ventricular systole to a local peak amplitude of the motion sensor coincident with ventricular diastole, or rate of onset, duration or magnitude of the local peak amplitude of the motion sensor coincident with ventricular diastole.

Example 61: The method of any of examples 48-60, wherein the period of time is greater than or equal to 10 seconds and less than or equal to 30 seconds.

Example 62: The method of any of examples 48-61, further comprising: monitoring, by the circuitry, one or more of electrical activity of the heart, impedance, or another electrical phenomenon via one or more of the plurality of electrodes.

Example 63: The method of example 62, further comprising: determining the indication of heart failure based on the detected motion of the implantable device during the period of time and the monitored signals.

Example 64: The method of any of examples 48-62, wherein the motion sensor is an accelerometer.

Example 65: The method of any of examples 48-64, wherein the target heart rate is greater than or equal to 100 beats per minute and less than or equal to 150 beats per minute.

Example 66: The method of any of examples 48-65, wherein the plurality of electrodes, the motion sensor, and the circuitry are included in an implantable medical device.

Example 67: The method of any of examples 48-66, the method further comprising outputting the determined indication of heart failure to a second device or network.

Example 68: The method of any of examples 48-67, wherein delivering the electrical stimulation therapy pacing protocol includes delivering pacing stimulation signals to the heart.

Example 69: The method of example 68, wherein delivering the electrical stimulation therapy pacing protocol further includes delivering the pacing stimulation signals to the heart at a plurality of atrioventricular intervals.

Example 70: A system including a plurality of electrodes; a motion sensor configured to detect motion; therapy generation circuitry electrically coupled to one or more of the plurality of electrodes; and processing circuitry configured to: control the therapy generation circuitry to deliver electrical pacing to a heart via one or more of the plurality of electrodes over a period of time at least one target heartrate; and determine an indication of heart failure based on a mean motion sensor amplitude detected during the period of time.

Example 71: The system of example 70, wherein the mean motion sensor amplitude is detected throughout the period of time.

Example 72: The system of any of examples 70-71, wherein the mean motion sensor amplitude is a root mean square amplitude.

Example 73: The system of example 72, wherein the root mean square amplitude is detected throughout the period of time.

Example 74: The system of example 70, wherein the processing circuitry is further configured to detect a mean motion sensor amplitude during multiple periods of time at the at least one target heartrate.

Example 75: The system of example 74, wherein the processing circuitry is further configured to determine an indication of heart failure based on mean motion sensor amplitude detected during the multiple periods of time.

Example 76: The system of example 74, wherein the processing circuitry is further configured to determine an indication of heart failure based on a trend in the mean motion sensor amplitude detected during the multiple periods of time.

Example 77: A method including delivering, by circuitry and via one or more of a plurality of electrodes, an electrical pacing to a heart over a period of time of at least a target heartrate; detecting, by a motion sensor, a mean motion sensor amplitude during the pacing protocol during the period of time; and determining, by the circuitry, an indication of heart failure based on the detected a mean motion sensor amplitude during the period of time.

Example 78: The method of example 77, the mean motion sensor amplitude is detected throughout the period of time.

Example 79: The method of any of examples 77-78, wherein the mean motion sensor amplitude is a root mean square amplitude.

Example 80: The method of example 79, wherein the root mean square amplitude is detected throughout the period of time.

Example 81: The method of example 77, the method further comprising: detecting a mean motion sensor amplitude during multiple periods of time at the at least one target heartrate.

Example 82: The method of example 81, the method further comprising: determining an indication of heart failure based on mean motion sensor amplitude detected during the multiple periods of time.

Example 83: The method of example 81, the method further comprising: determining an indication of heart failure based on a trend in the mean motion sensor amplitude detected during the multiple periods of time.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

1. A system comprising:
a plurality of electrodes;
a motion sensor configured to detect motion;
therapy generation circuitry electrically coupled to one or more of the plurality of electrodes; and
processing circuitry configured to:
control the therapy generation circuitry to deliver an electrical stimulation therapy pacing protocol to a heart via one or more of the plurality of electrodes over a period of time to increase heartrate during the period of time to at least a target heartrate; and
determine an indication of heart failure based on the detected motion during the period of time.

2. The system of claim 1, wherein the indication of heart failure is one or more of an extent of heart failure, a form of heart failure, a presence or absence of heart failure, a possibility of heart failure, or a change in heart failure severity.

3. The system of claim 1, wherein to determine an indication of heart failure based on the detected motion during the period of time includes:
determine a feature of heart motion of the heart based on the detected motion;
determine a difference between the determined feature of heart motion and a respective feature of heart motion threshold; and
determine the indication of heart failure based on the determined difference.

4. The system of claim 1, wherein to determine an indication of heart failure based on the detected motion during the period of time includes:
determine a change in value of a feature of heart motion of the heart based on the detected motion;
determine a difference between the determined change in value of a feature of heart motion and a respective change threshold; and
determine the indication of heart failure based on the determined difference.

5. The system of claim 4, wherein the change in value is between the feature of heart motion at a resting rate and the feature of heart motion at an elevated pacing rate.

6. The system of claim 3, wherein the feature of heart motion is a mean motion sensor amplitude throughout the period of time.

7. The system of claim 3, wherein the feature of heart motion includes one or more of a local peak amplitude of the motion sensor signal coincident with ventricular systole, a local peak amplitude of the motion sensor signal coincident with an S1 heart sound, or a duration or rate of onset of a local peak amplitude of the motion sensor signal coincident with an S1 heart sound.

8. The system of claim 3, wherein the feature of heart motion includes one or more of:
rate of onset of a local peak amplitude of the motion sensor coincident with ventricular systole, or
duration of the local peak amplitude of the motion sensor coincident with ventricular systole.

9. The system of claim 3, wherein the feature of heart motion includes one or more of:
an interval from a local peak amplitude of the motion sensor signal coincident with ventricular systole to a local peak amplitude of the motion sensor coincident with ventricular diastole, or
rate of onset, duration or magnitude of the local peak amplitude of the motion sensor coincident with ventricular diastole.

10. The system of claim 1, further comprising sensing circuitry configured to monitor signals from one or more of the plurality of electrodes to monitor one or more of electrical activity of the heart, impedance, or another electrical phenomenon,
wherein the processing circuitry is configured to determine the indication of heart failure based on the detected motion during the period of time and the monitored signals.

11. A system comprising:
a plurality of electrodes;
a motion sensor configured to detect motion;
therapy generation circuitry electrically coupled to one or more of the plurality of electrodes; and
processing circuitry configured to:
determine, based on the detected motion, a heartrate of a heart is at least a target threshold;
in response to determining the heartrate is at least the target threshold, control the therapy generation circuitry to deliver, via one or more of the plurality of electrodes, cardiac pacing to the heart at a plurality of atrioventricular intervals over a period of time; and
determine an indication of heart failure based on the detected motion during the period of time.

12. The system of claim 11, wherein to determine an indication of heart failure based on the detected motion during the period of time includes:
determine a feature of heart motion of the heart based on the detected motion during the period of time;
determine a difference between the feature of heart motion and a respective feature of heart motion threshold; and
determine the indication of heart failure based on the determined difference.

13. The system of claim 11, wherein to determine an indication of heart failure based on the detected motion during the period of time includes:
determine a change in value of a feature of heart motion of the heart based on the detected motion during the period of time;
determine a difference between the determined change in value of a feature of heart motion and a respective change threshold; and
determine the indication of heart failure based on the determined difference,
wherein the change in value is between the feature of heart motion at a resting rate and the feature of heart motion at an elevated pacing rate.

14. The system of claim 12, wherein the feature of heart motion is a mean motion sensor amplitude throughout the period of time.

15. The system of claim 12, wherein the feature of heart motion includes one or more of a local peak amplitude of the motion sensor signal coincident with ventricular systole, a local peak amplitude of the motion sensor signal coincident with an S1 heart sound, or a duration or rate of onset of a local peak amplitude of the motion sensor signal coincident with an S1 heart sound.

16. The system of claim 12, wherein the feature of heart motion includes one or more of:
rate of onset of a local peak amplitude of the motion sensor coincident with ventricular systole, or
duration of the local peak amplitude of the motion sensor coincident with ventricular systole.

17. A method comprising:
delivering, by circuitry and via one or more of a plurality of electrodes, an electrical stimulation therapy pacing protocol to a heart to increase heartrate during a period of time to at least a target heartrate;
detecting, by a motion sensor, motion during the pacing protocol during the period of time; and
determining, by the circuitry, an indication of heart failure of the heart based on the detected motion during the period of time.

18. The method of claim 17, wherein the indication of heart failure is one or more of an extent of heart failure, a form of heart failure, a presence or absence of heart failure, a possibility of heart failure, or a change in heart failure severity.

19. The method of claim 17, wherein determining an indication of heart failure based on the detected motion during the period of time includes:
determining a feature of heart motion of the heart based on the detected motion;
determine a difference between the determined feature of heart motion and a respective feature of heart motion threshold; and
determine the indication of heart failure based on the determined difference.

20. The method of claim 17, wherein determining an indication of heart failure based on the detected motion during the period of time includes:
determining a change in value of a feature of heart motion of the heart based on the detected motion;
determining a difference between the determined change in value of a feature of heart motion and a respective change threshold; and
determining the indication of heart failure based on the determined difference.
